(19) United States
(12) Patent Application Publication
(10) Pub. No.: US 2012/0253400 A1
(43) Pub. Date: Oct. 4, 2012

(54) VERTEBRAL COLUMN IMPLANT

(75) Inventors: Jonathan CLARK, Kloten (CH); Reto Braunschweiler, Neftenbach (CH); Thomas Zehnder, Bach (CH)

(73) Assignee: Corporation Spinelab AG, Winterthur (CH)

(21) Appl. No.: 13/431,087

(22) Filed: Mar. 27, 2012

(30) Foreign Application Priority Data
Mar. 31, 2011 (EP) 11160692.7

(51) Int. Cl. A61B 17/70

(52) U.S. Cl. 606/264

(57) ABSTRACT

A vertebral column implant for stabilizing and stiffening of vertebral bodies of a vertebral column comprises at least one first and at least one second bone screw, which is screwable into a vertebral body. A first rigid connecting element is insertable into first receiving means of the first bone screw. A second elastic connecting element is insertable into second receiving means of the second bone screw. The connecting means are installed on a further bone screw and comprise a first and second clamping device for respectively holding tightly the first and second connecting element. The first clamping device has a U-shaped receiving part for the end region of the first connecting element, which is designed spherical, a clamping means being placeable on the U-shaped receiving part, with which clamping means the spherical end region is able to be clamped against the bottom surface of the U-shaped receiving part.

This invention relates to a vertebral column implant for stabilization and stiffening of vertebral bodies of a vertebral column, comprising at least one first bone screw having a screw-in part that is screwable into a vertebral body, and first receiving means; at least one second bone screw having a screw-in part that is screwable into a vertebral body, and second receiving means; at least one first connecting element, which is rigid and is insertable into the first receiving means of the first bone screw and is able to be fixed therein; at least one second connecting element, which is elastic and is insertable into the second receiving means of the second bone screw and is able to be fixed therein; and connecting means, with which in each case a first connecting element is connectible to a second connecting element.

Such vertebral column implants for stabilization and stiffening of vertebral bodies of a vertebral column are known in diverse designs. Achieved with the stiffening part of this vertebral column implant is that an osseous growing together between the vertebral bodies stiffened in this way takes place, which leads to a desired stabilization of the vertebral column, while, with the elastic region of the vertebral column implant, the vertebral bodies are stabilized in a supporting way. A certain movability between the individual vertebral bodies is desired and is permitted.

Such a vertebral column implant for stabilization and stiffening of vertebral bodies of a vertebral column is known for example from EP-A-1 961 392.

With these known vertebral column implants, the placement of the bone screws in the individual vertebral bodies, especially in the region of the transition from the elastic connecting element to the rigid connecting element, must take place very precisely. Certain instances of imprecision can be corrected in that the rigid connecting element is suitably bent, which must be accomplished during the insertion of the vertebral column implant in a vertebral column, and which makes the procedure more difficult.

The object of the present invention thus consists in creating a vertebral column implant for stabilization and stiffening of vertebral bodies of a vertebral column which is of simple construction, and thereby the insertion and connection of the rigid and elastic connecting elements is simplified, and in which in particular the first connecting element, which is rigid, can be aligned with respect to the second elastic connecting element on all sides in a certain angular range.

The object is achieved according to the invention in that the connecting means are installed on a further bone screw and comprise a first clamping device for holding tightly the first connecting element and a second clamping device for holding tightly the second connecting element, and the first clamping device has a U-shaped receiving part for the end region of the first connecting element, which end region is designed spherical, and a clamping means is placeable on the U-shaped receiving part, with which clamping means the spherical end region is able to be clamped against the bottom surface of the U-shaped receiving part.

Through this design, the first rigid connecting element can be aligned with respect to the first clamping device and thus with respect to the second elastic connecting element on all sides in a certain angular range, and can be adapted in an optimal way to the bone screws inserted in the vertebral bodies. Achieved thereby is that an imprecise placement of the bone screws in the vertebral bodies can be compensated for through the possibility of a polyaxial alignment of the first connecting element instead of through complicated and difficult bending of the rigid connecting element during the procedure.

Preferably the bottom surface of the U-shaped receiving part has a concave spherical shape that corresponds to the spherical end region of the first connecting element, whereby an optimal pressing and consequently fixing of the spherical end region of the first connecting element in the first clamping device is obtained.

Preferably the surface of the clamping means turned toward the spherical end region of the first connecting element also has a concave spherical recess, whereby the firm holding and fixing of the spherical end region in the first clamping device is additionally improved.

Preferably the clamping means comprises a clamping screw which is screwable into a threading made in the U-shaped receiving part, which results in a simple design for the clamping means.

Preferably the clamping means comprises a pressing element, which is insertable between the clamping screw and the spherical end region of the first connecting element. Thereby achieved is that the clamping screw does not come into direct contact with the spherical end region of the first connecting element, which prevents damage to this spherical end region during the clamping procedure, and thus no chips or shavings are generated.

Preferably the clamping screw is connected in a rotatable way to the pressing element, whereby it is achieved that the clamping screw and the pressing element can be inserted as a unit into the U-shaped receiving part, which simplifies this insertion procedure.

Another advantageous embodiment of the invention consists in that the second clamping device has a further U-shaped receiving part for the end region of the second connecting element, on which receiving part a further clamping means is placeable with which the end region of the second connecting element is held in the further U-shaped receiving part. The two connecting elements can thereby be inserted in the connecting means and clamped independently of one another.

Preferably the surface of the further clamping means turned toward the end region of the second connecting element and/or the further U-shaped receiving part are provided with structures. Obtained thereby is an improved connection between the second elastic connecting element and the further clamping means and/or the bottom surface of the further U-shaped receiving part.

Preferably at least the end region of the second connecting element is provided with further structures which are designed in a way complementary to the structures of the further U-shaped receiving part, whereby a formfitting connection is obtained between the second connecting element and the further U-shaped receiving part.

Preferably the structures and the further structures consist of ribs and grooves which are aligned substantially transversely to the longitudinal axis of the second connecting element, which ribs and grooves can be easily produced.

An embodiment of the invention will be explained more closely in the following, by way of example, with reference to the attached drawings.

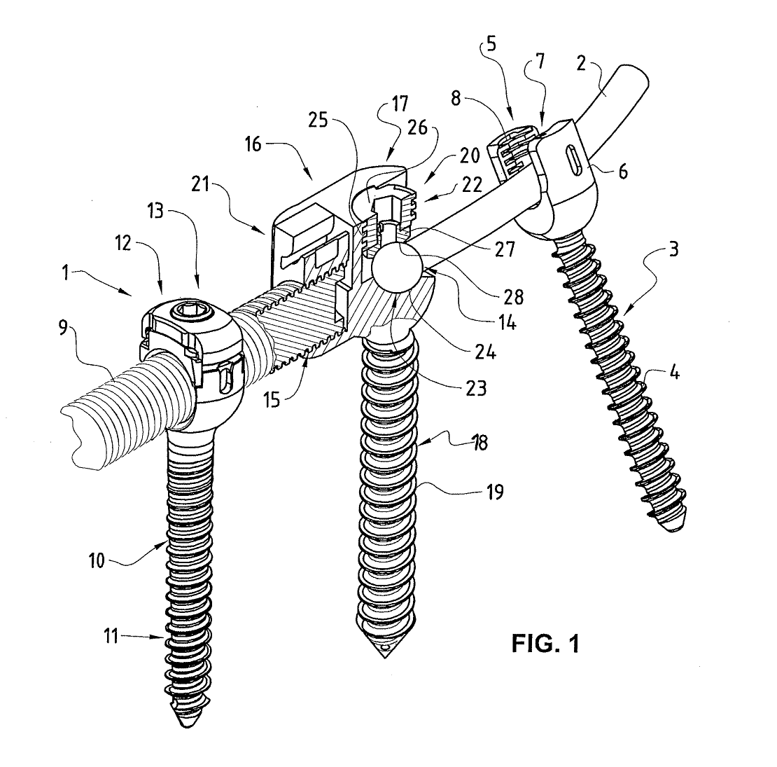

FIG. 1 shows in a three-dimensional representation a vertebral column implant, which comprises a first rigid connecting element, a second elastic connecting element and bone screws, in which the connecting elements are inserted and held, and a further bone screw, in which the end regions of the two connecting elements are inserted and fixed;

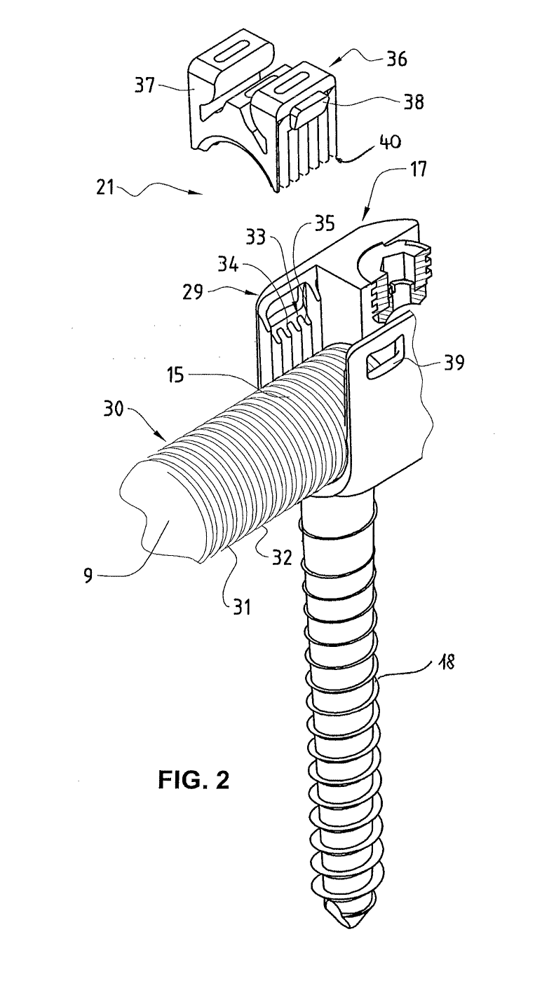

FIG. 2 shows in a three-dimensional representation the second clamping device installed in the further bone screw for holding tightly the second connecting element; and

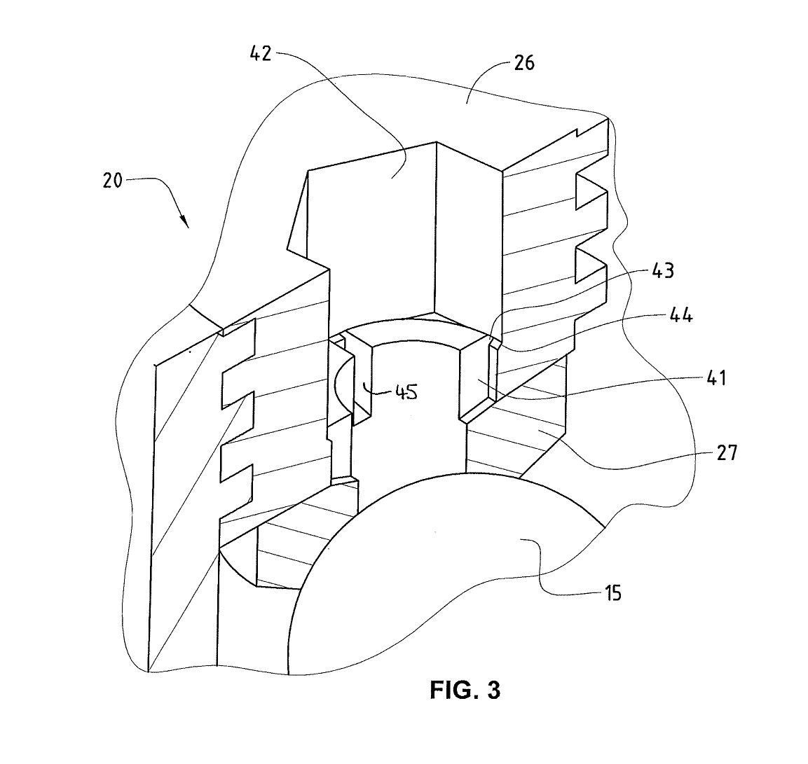

FIG. 3 is an enlarged sectional view of the clamping means with the pressing element, which are inserted in the U-shaped receiving part of the further bone screw.

The vertebral column implant 1 spatially represented in FIG. 1 has a first connecting element 2, which is rigid. This first connecting element 2 is held in a first bone screw 3, which comprises in a known way a screw-in part 4 that can be screwed into a vertebral body in a way likewise known (not shown). This first bone screw 3 is provided with first receiving means 5 for holding the first connecting element 2, which first receiving means are designed as U-shaped receiving part 7 in the head part 6 of the first bone screw 3. The first connecting element 2, which is designed as a rod, can be placed in this U-shaped receiving part 7. The U-shaped receiving part 7 has on the inside a threading 8, into which a clamping screw can be screwed in a known way (not shown), by means of which clamping screw the first connecting element 2 is clamped in the U-shaped receiving part 7.

The vertebral column implant 1 shown here further comprises a second connecting element 9, which is elastic and which is held in a second bone screw 10. This second bone screw 10 likewise comprises a screw-in part 11 that can be screwed into a vertebral body in a known way (not shown). Disposed in the head part 12 of this second bone screw 10 are second receiving means 13, in which the second connecting element 9 can be fixed and held in the second bone screw 10. Such a second bone screw with the second receiving means for receiving the second connecting element is described in detail in the European patent application having the publication number 2 074 957, which is hereby referred to.

As can be seen from FIG. 1, the end region 14 of the first connecting element 2 and the end region 15 of the second connecting element 9 are held in a connecting way in connecting means 16. These connecting means 16 are disposed in the head part 17 of a further bone screw 18, which further bone screw 18 is likewise provided with a screw-in part 19 with which this further bone screw can also be screwed into a vertebral body in a known way (not shown).

As can be seen furthermore from FIG. 1, the connecting means 16, which are installed in the head part 17 of the further bone screw 18, comprise a first clamping device 20 for holding tightly the first connecting element 2 and a second clamping device 21 for holding tightly the second connecting element 9, which will be described later in detail. The first clamping device 20 is provided with a U-shaped receiving part 22, in which the end region 14 of the first connecting element 2 can be inserted. The end region 14 of the first connecting element 2 is designed spherical. The bottom surface 23 of the U-shaped receiving part 22 has a concave spherical shape 24, which corresponds to the spherical end region 14 of the first connecting element 2.

Provided in the U-shaped receiving part 22 is a threading 25, into which a clamping screw 26 can be screwed. In the embodiment example shown here, a pressing element 27 is disposed between the clamping screw 26 and the spherical end region 14 of the first connecting element 2 inserted in the U-shaped receiving part 22. In a known way, the one surface of the pressing element 27 supports itself on the clamping screw 26. The surface of this pressing element 27 turned toward the spherical end region 14 of the first connecting element 2 has a concave spherical recess 28 corresponding to the spherical end region 14 of the first connecting element 2.

In the state of the clamping screw being screwed into the threading 25 and tightened, this clamping screw 26 thus presses on the pressing element 27; the concave spherical recess 28 is pressed on the spherical end region 14 of the first connecting element 2. This spherical end region 14 of the first connecting element 2, in turn, is thereby pressed on the concave spherical shape 24 of the bottom surface 23 of the U-shaped receiving part 22; the first connecting element 2 is thereby held in an optimal way in the connecting means 16 of the further bone screw 18. Through the spherical design of the end region 14 of the first connecting element 2 and the corresponding concave spherical receiving areas 24 and 28 of the first clamping device 20, the first connecting element 2 can be aligned angularly before tightening of the clamping screw 26, so that it can be polyaxially aligned in an optimal way with respect to the first bone screw 3. The insertion of a first connecting element 2 in the provided vertebral column implant is thereby very much simplified. Thus an alignment by bending the first connecting element 2 during insertion of the vertebral column implant in a vertebral column to be stabilized is not necessary.

As can be seen from FIG. 2, the second clamping device 21, which is installed in the head part 17 of the further bone screw 18, has a further U-shaped receiving part 29. The end region 15 of the second connecting element 9 can be inserted in this further U-shaped receiving part 29. At least this end region 15 of the second connecting element 9 is provided with structures 30, which in the embodiment example shown here are designed as ribs and grooves 31, 32 that are aligned substantially transversely to the longitudinal axis of the second connecting element 9. As can be seen from FIG. 2, the further U-shaped receiving part 29 is provided with further structures 33, which are likewise designed as ribs 34 and grooves 35, which correspond to the ribs 31 and grooves 32 of the second connecting element 9. Through this design, a formfitting connection is obtained between the second clamping device 21 and the end region 15 of the second connecting element 9. Of course it would also be conceivable to provide other structural forms. For instance, these could be provided only in the further U-shaped receiving part 29, which structural forms penetrate into the surface of the end region 15 of the second connecting element 9 when it is inserted into this further U-shaped receiving part 29, whereby a practically formfitting connection is likewise obtained.

The further U-shaped receiving part 29 can be closed off by a further clamping means 36, which, in the embodiment example shown here, is constituted in a known way by an insert part 37, which is made of an elastic material, and which is provided with snap elements 38 that, in inserted state, can snap into apertures 39 provided in the further U-shaped receiving part 29, and can hold the insert part 37 in this position. The insert part 37 can also be provided with structures 40, which are able to co-operate with the structures 30 of the end region 15 of the second connecting element 9 and with the further structures 33 in the further U-shaped receiving part 29.

This further clamping means 36 could also be constituted by a clamping screw that could be screwed into a threading correspondingly provided in the further U-shaped receiving part 29. Of course other known and suitable clamping devices would also be conceivable.

As can be seen from the enlarged view according to FIG. 3, the clamping screw 26, which is screwed into the first clamping device 20 (FIG. 1), and the inserted pressing element 27 can be connected to one another in a rotatable way. For this purpose the pressing element 27 has a projection 41, which projects into a hole 42 of the clamping screw 26, and which is provided with a protrusion 43. A slot 44 can be made in the hole 42, into which slot the protrusion 43 can penetrate. The projection 41 is provided with openings 45, so that this projection 41 can be pressed into the hole 42. When the protrusion 43 reaches the slot 44, an elastic tension release takes place. The pressing element 27 is thereby connected to the clamping screw 26 in a rotatable way, which has as a result a simplified handling during insertion of the vertebral column implant. The protrusion 43 and the slot 44 can be provided with play, so that no forces arise here during tightening of the clamping screw 26 and pressing of the pressing element 27.

The elastic second connecting element used in vertebral column implants of this kind is preferably made of a polyurethane-based, biocompatible synthetic material, while the other parts are made of a titanium alloy. Of course other suitable materials can also be used.

Embodiments have been described in the preceding that have in each case been composed of a total of three screws, the first and the middle screw being connected to one another via a rigid connecting element, and the middle screw and the second screw being connected to one another via an elastic connecting element. Of course many other embodiment examples are conceivable having more screws, in which embodiments the rigid and elastic connecting elements extend over more than two screws, for example. It is also conceivable for more than two connecting element parts to be used; for instance, a rigid connecting element could be inserted between two elastic connecting elements, the previously described further bone screws being inserted in the region of the connections of these connecting elements. Thus a vertebral column implant put together in any desired way, depending upon need, can be used.

Obtained with this invention is a vertebral column implant for stabilization and stiffening of vertebral bodies of a vertebral column which can be adapted in an optimal way to the circumstances, which makes possible in an optimal way a polyaxial adaptation of a rigid connecting element with respect to an elastic connecting element, and which is easy to handle.

1. A vertebral column implant for stabilization and stiffening of vertebral bodies of a vertebral column, comprising:
at least one first bone screw having a screw-in part that is screwable into a vertebral body, and first receiving means;
at least one second bone screw having a screw-in part that is screwable into a vertebral body, and second receiving means;
at least one first connecting element, which is rigid and is insertable into the first receiving means of the first bone screw and is able to be fixed therein;
at least one second connecting element, which is elastic and is insertable into the second receiving means of the second bone screw and is able to be fixed therein; and
connecting means, with which in each case a first connecting element is connectible to a second connecting element,
wherein the connecting means are installed on a further bone screw and comprise a first clamping device for holding tightly the first connecting element and a second clamping device for holding tightly the second connecting element, and the first clamping device has a U-shaped receiving part for the end region the first connecting element, which end region is designed spherical, and a clamping means is placeable on the U-shaped receiving part, with which clamping means the spherical end region is able to be clamped against the bottom surface of the U-shaped receiving part.

2. The vertebral column implant according to claim 1, wherein the bottom surface of the U-shaped receiving part has a concave spherical shape that corresponds to the spherical end region of the first connecting element.

3. The vertebral column implant according to claim 1, wherein the surface of the clamping means turned toward the spherical end region of the first connecting element has a concave spherical recess.

4. The vertebral column implant according to claim 1, wherein the clamping means comprises a clamping screw which is screwable into a threading made in the U-shaped receiving part.

5. The vertebral column implant according to claim 4, wherein the clamping means comprises a pressing element, which is insertable between the clamping screw and the spherical end region of the first connecting element.

6. The vertebral column implant according to claim 5, wherein the clamping screw is connected in a rotatable way to the pressing element.

7. The vertebral column implant according to claim 1, wherein the second clamping device has a further U-shaped receiving part for the end region of the second connecting element, on which receiving part a further clamping means is placeable with which the end region of the second connecting element is held in the further U-shaped receiving part.

8. The vertebral column implant according to claim 7, wherein the surface of the further clamping means turned toward the end region of the second connecting element and/or the further U-shaped receiving part are provided with structures

9. The vertebral column implant according to claim 7, wherein at least the end region of the second connecting element is provided with structures which are designed in a way complementary to the further structures of the further U-shaped receiving part

10. The vertebral column implant according to claim 8, wherein the structures and the further structures consist of ribs and grooves which are aligned substantially transversely to the longitudinal axis of the second connecting element.
